(19) United States
(12) Patent Application Publication
(10) Pub. No.: US 2012/0121708 A1
(43) Pub. Date: May 17, 2012

(54) Acetazolamide Microparticle And Its Preparation Method And Use

(75) Inventors: Yan-Ping Chen, Taipei (TW); Feng-Nien Tsai, Taipei (TW)

(73) Assignee: National Taiwan University, Taipei (TW)

(21) Appl. No.: 12/946,793

(22) Filed: Nov. 15, 2010

(51) Int. Cl. A61K 9/14; A61P 27/06; A61P 43/00; C07D 285/135; A61P 7/10; A61P 13/02; B32B 5/16; A61K 31/433; A61P 25/08

(52) U.S. Cl. 424/489; 514/363; 428/402; 548/140

(57) ABSTRACT

A method for preparing an acetazolamide microparticle having a mean particle size ranged between 0.36 μm and 18 μm is provided. The method includes steps of dissolving an acetazolamide in a solvent to form an acetazolamide solution; and mixing the acetazolamide solution with a supercritical fluid at a temperature and a pressure above a critical point of the supercritical fluid for forming the acetazolamide microparticle, wherein the solvent is miscible with the supercritical fluid.

### FIELD OF THE INVENTION

The present invention relates to an acetazolamide microparticle, its preparation method and use thereof, particularly to an acetazolamide microparticle obtained by a supercritical anti-solvent (SAS) process and its pharmaceutical applications.

### BACKGROUND OF THE INVENTION

Acetazolamide is a drug that is a carbonic anhydrase inhibitor and is used to reduce ocular tension and treating glaucoma, epileptic seizures, benign intracranial hypertension (pseudotumor cerebri), altitude sickness, cystinuria, and dural ectasia. Acetazolamide is also used as a diuretic. The commercial acetazolamide drug usually has a particle size of about 20 μm.

Micronization is an important process for the pharmaceutical industry and there have been developed many related technologies. Traditional physical micronization techniques are based on friction to reduce particle size. Such methods include crushing, cutting, milling and grinding. However, during the processes of crushing, cutting, milling or grinding, the wear or exfoliation of the tool or machine used to implement the above processes may contaminate the drugs. Further, during the processes of reducing the particle size by the mechanical force, the original crystal face and form of the drugs may be destroyed, which may affect the efficacy and stability of the physical and chemical properties of the drugs. Traditional chemical micronization techniques are acomplished by evaporation, heating and cooling, or adding an ingredient in the solution to reduce the solubility of the medication solute in a solution, and thereby the crystalline or amorphous particles are formed due to the saturation and the deposition of the medication solute. However, the drug particles obtained by such chemical methods do not have a specific and narrow range of the particle size distribution and could have different crystal forms, and there might be the problem regarding the residual solvent on the formed particles. Therefore, it is important to provide a technich for preparing the drug particals where the particle size, distribution, and crystal properties could be effectively controlled and the properties of the drug are maintained stable.

Hence, because of the defects in the prior arts, the inventors provide an acetazolamide microparticle, its preparation method and use thereof to effectively overcome the demerits existing in the prior arts.

### SUMMARY OF THE INVENTION

The present invention is related to a SAS process for precipitating particles with supercritical fluids and applications of the prepared particles. The SAS processes can be used to precipitate particles of a substance that is insoluble in the supercritical fluid, provided that the supercritical fluid is miscible with the liquid in which the substance is dissolved.

One purpose of the present invention is to provide an acetazolamide microparticle, which has a mean particle size ranged between 0.36 μm and 18 μm.

In accordance with another aspect of the present invention, a method for preparing an acetazolamide microparticle having a mean particle size ranged between 0.36 μm and 18 μm is provided. The method comprises steps of: dissolving an acetazolamide in a solvent to form an acetazolamide solution; and mixing the acetazolamide solution with a supercritical fluid at a temperature and a pressure above a critical point of the supercritical fluid for forming the acetazolamide microparticle, wherein the solvent is miscible with the supercritical fluid.

In accordance with a further aspect of the present invention, a method for treating a disease being one selected from a group consisting of a diuresis, a high ocular pressure, a glaucoma, a high altitude disease, an epilepsy and an edema is provided. The method comprises a step of administering to a subject in need thereof a pharmaceutical composition comprising an acetazolamide microparticle having a mean particle size ranged between 0.36 μm and 18 μm.

The above objects and advantages of the present invention will become more readily apparent to those ordinarily skilled in the art after reviewing the following detailed descriptions and accompanying drawings, in which:

### BRIEF DESCRIPTION OF THE DRAWINGS

FIG. 1 is a diagram showing the operation of the supercritical anti-solvent (SAS) method provided in the present invention.

FIGS. 2(a)-(d) are diagrams showing the SEM results of the acetazolamide bulk drug and the acetazolamide microparticles obtained from the embodiments 1 to 3.

FIG. 3 is a diagram showing the comparisons of the particle sizes and distributions between the embodiments 1 to 3.

FIGS. 4(a)-(d) are diagrams showing the differential scanning calorimetry (DSC) analyses results of the acetazolamide bulk drug and the acetazolamide microparticles obtained from embodiments 1-3.

FIGS. 5(a)-(c) are diagrams showing the SEM results of the acetazolamide microparticles obtained from the embodiments 4 to 6.

FIG. 6 is a diagram showing the comparisons of the particle sizes and distributions between the embodiments 4 to 6.

FIGS. 7(a)-(c) are diagrams showing the SEM results of the acetazolamide microparticles obtained from the embodiments 7 to 9.

FIG. 8 is a diagram showing the comparisons of the particle sizes and distributions between the embodiments 7 to 9.

FIGS. 9(a)-(b) are diagrams showing the SEM results of the acetazolamide microparticles obtained from the embodiments 10 and 11.

FIGS. 10(a)-(b) are diagrams showing the SEM results of the acetazolamide microparticles obtained from the embodiments 12 and 13.

FIG. 11 is a curve diagram showing the dissolution rates of the acetazolamide bulk drug, and the acetazolamide microparticles obtained from embodiments 11 and 1 of the present invention.

### DETAILED DESCRIPTION OF THE PREFERRED EMBODIMENT

The present invention will now be described more specifically with reference to the following embodiments. It is to be noted that the following descriptions of preferred embodiments of this invention are presented herein for the purposes of illustration and description only; it is not intended to be exhaustive or to be limited to the precise form disclosed.

The present invention relates to an acetazolamide microparticle, which has a mean particle size smaller than 18 μm, preferably smaller than 15 μm, more preferably smaller than 10 μm, further preferably smaller than 5 μm, and best smaller than 1 μm. Specifically, the particle size of the acetazolamide microparticle in the present invention is apparently smaller than that of the acetazolamide bulk drug, which substantially increases the bioavailability of the acetazolamide drug.

In addition, the acetazolamide microparticles obtain from the present invention could have different crystal forms and shapes. For example, the crystal form of the acetazolamide microparticle of the present invention could be Form II with the regular rod-like crystal shape or Form I with the irregular crystal shape. It is found via the dissolution rate test that compared with the Form I acetazolamide microparticle, the Form II acetazolamide microparticle has a higher dissolution rate.

The present invention further relates to a method for preparing acetazolamide microparticles via the supercritical fluids. The method comprises the step of mixing an acetazolamide solution and a supercritical fluid for forming the acetazolamide microparticles, wherein the solvent in the acetazolamide solution is miscible with the supercritical fluid.

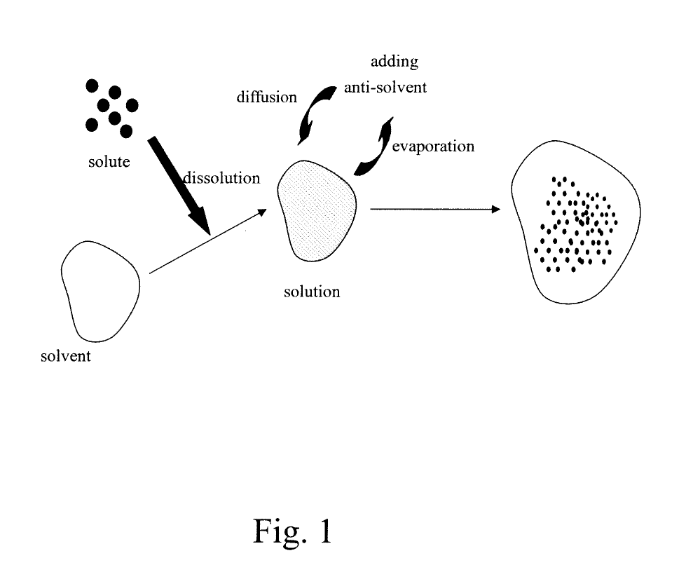

Please refer to FIG. 1, which is a diagram showing the operation of the supercritical anti-solvent (SAS) method provided in the present invention. In brief, the supercritical fluid is serving as an anti-solvent causing the supersaturation of the solution and leading to the nucleation and precipitation of the microparticles, i.e. the acetazolamide microparticles, with a desired particle size, and the separation of the yielded microparticles from the solution is achieved by the vaporization.

It is to be understood that the suitable supercritical fluids and solvents could be selected based on the principles that the SAS is possible only if the liquid solvent is completely miscible with the supercritical fluid and if the solute is insoluble in this mixture. Carbon dioxide is the most widely used supercritical fluid because of its relatively low critical temperature (31° C.) and pressure (74 bar). In addition, the supercritical CO₂ is non-toxic, non-flammable, inexpensive, and has GRAS (generally regarded as safe) status. Therefore, carbon dioxide is selected to provide the supercritical fluid in a preferred embodiment of the present invention.

According to the method of the present invention, firstly, the acetazolamide bulk drug to be micronized is dissolved in a solvent to form an acetazolamide solution. The selection of the abovementioned solvent is based on that the solvent is completely miscible with the used supercritical fluid while the acetazolamide is insoluble in the supercritical fluid. In the method of the present invention, the solvent could be selected from a group consisting of methanol, ethanol, methylene chloride, N-methyl-pyrrolidone (NMP), ethyl acetate, acetone and a combination thereof. For achieving the purpose of the micronization, the solvent is preferably selected from ethyl acetate, acetone and a combination thereof, and more preferably is ethyl acetate.

Since different solvents show different affinities to the acetazolamide, it is found the acetazolamide microparticles with different crystal forms and/or particle sizes could be obtained by adopting different solvents. When ethyl acetate or acetone is used as the solvent, the acetazolamide microparticle with the crystal form of Form II will be obtained, and when ethanol is used, the acetazolamide microparticle with the crystal form of Form I will be obtained.

Any suitable manner could be used to mix the acetazolamide solution with the supercritical fluid. In one embodiment according to the method of the present invention, the mixing procedure is performed by delivering the acetazolamide solution into a container including the supercritical fluid. Specifically, the supercritical fluid is fed into the container from the top thereof first so that the container is filled with the supercritical fluid, and the supercritical fluid is kept fluid at a constant flow rate. Then, the acetazolamide solution is fed from the top into the container. This is called a continuous mixing method. That is, in this embodiment, the acetazolamide solution and the supercritical fluid are fed into the container in the manner of the concurrent flow.

If in the method of the present invention, a batch mixing method where the supercritical fluid is introduced into the static solution is adopted, it is hard to mix the supercritical fluid with the solution evenly because of the limited agitation caused during the mixing procedure. For the batch mixing method, there would be a high mass transfer resistance existing between the supercritical fluid and the solution, which would cause an insufficient number of nuclei and thus a bigger particle size since most solute is attached on the limited nuclei and accelerates the formation of the crystals. On the other hand, as to the continuous mixing method, since it is relatively easy to mix the solution and the supercritical fluid evenly, and the equilibrium solubility of the acetazolamide in the mixed solution is low, a large number of the nuclei are formed due to the achieved high super-saturation. That is to say, the mass transfer rate of the supercritical fluid is higher than the nucleation and growth rate of the crystals. Therefore, the continuous mixing method is advantageous in preparing the microparticles with the small particle size and narrow particle size distribution range.

It is found in the present invention that the solution concentration has a competitive effect on the micronization. Further, under the low solution flow rate condition, the average particle size increases with the increasing solution concentration; and under the high solution flow rate condition, the average particle size decreases with the increasing solution concentration. Generally, when CO₂ is used as the supercritical fluid, the acetazolamide solution concentration is at least 10% of the saturated concentration, preferably is at least 25% of the saturated concentration, more preferably is at least 50% of the saturated concentration, and best is at least 75% of the saturated concentration. In addition, when the flow rate of the supercritical CO₂ is ranged from 2 l/min to 4 l/min, the flow rate of the acetazolamide solution is generally ranged from 0.1 ml/min to 5 ml/min, and preferably ranged from 0.8 ml/min to 1.5 ml/min.

Further, it is found in the present invention that either the mixing pressure or the temperature has a competitive effect on the micronization. When CO₂ is used as the supercritical fluid, during the mixing procedure, the pressure and the temperature are usually ranged from 80 Pa to 160 Pa and 20° C. to 70° C. , respectively, and preferably ranged from 90 Pa to 110 Pa and 30° C. to 45° C., respectively.

One embodiment of the method according to the present invention further comprises a purification step for removing the residual solvent and thereby improving the quality of the obtained acetazolamide microparticles. The purification step could be achieved by continuously letting the supercritical fluid pass through the formed acetazolamide microparticle.

The present invention also relates to an application of manufacturing drugs by using the acetazolamide microparticle of the present invention, wherein the drugs are used for treating a diuresis, a high ocular pressure, a glaucoma, a high altitude disease, an epilepsy and/or an edema.

Supercritical CO₂ is taken as an example for specifying the operation conditions of the SAS method and the results thereof. However, such example is used to exemplify rather than limiting the present invention.

Operation and Analysis Methods

The saturated solubility of the acetazolamide is tested by the known technologies in this field. The microparticle morphology and size are detected by the scanning electron microscopy (SEM). The particle size distribution (PSD) is analyzed by the image analysis software “Image J”. The crystal properties are analyzed by the X-Ray diffractometer. The changes in the crystal form are detected by the differential scanning calorimetry (DSC). The qualitative analyses of the acetazolamide bulk drug and the acetazolamide microparticle of the present invention are performed by the Fourier transform infrared spectrometer (FTIR). The analyses of the dissolution rate are performed by the dissolution tester.

### [Embodiments 1 to 3] Solvent Effect

The solvent effect is studied under the fixed mixing pressure, mixing temperature, solution concentration and solution flow rate and different solvents. The operation parameters of the embodiments 1 to 3 are shown in Table 2, wherein the used solvents are ethanol (embodiment 1), acetone (embodiment 2) and ethyl acetate (embodiment 3). The mean particle size of the acetazolamide bulk drug is 19.64±13.2 μm. The saturated solubility of the acetazolamide is 1.5 mg/ml (in ethanol), 8.3 mg/ml (in acetone) or 0.6 mg/ml (in ethyl acetate). With the existing ethanol, the solubility of the acetazolamide in the supercritical CO₂ is 5.7×10⁻⁶ mg/ml (T=40° C. and P=150 Pa).

TABLE 2

| | | solution | Conc. | | | mean | | |
|---|---|---|---|---|---|---|---|---|
| | | FR | (% of the | | | particle | | |
| embodiment | solvent | (ml/min) | sat. conc.) | T(° C.) | P(Pa) | size (μm) | SE(μm) | R(%) |
| 1 | ethanol | 1 | 30 | 35 | 100 | 4.95 | 2.97 | 10.78 |
| 2 | acetone | 1 | 30 | 35 | 100 | 0.86 | 0.45 | 84.49 |
| 3 | ethyl | 1 | 30 | 35 | 100 | 0.73 | 0.34 | 60.81 |
| | acetate | | | | | | | |

Abbreviations: FR, flow rate; Conc., concentration; Sat. conc., saturated concentration; T, temperature; P, pressure; SE, standard error; R, recovery rate.

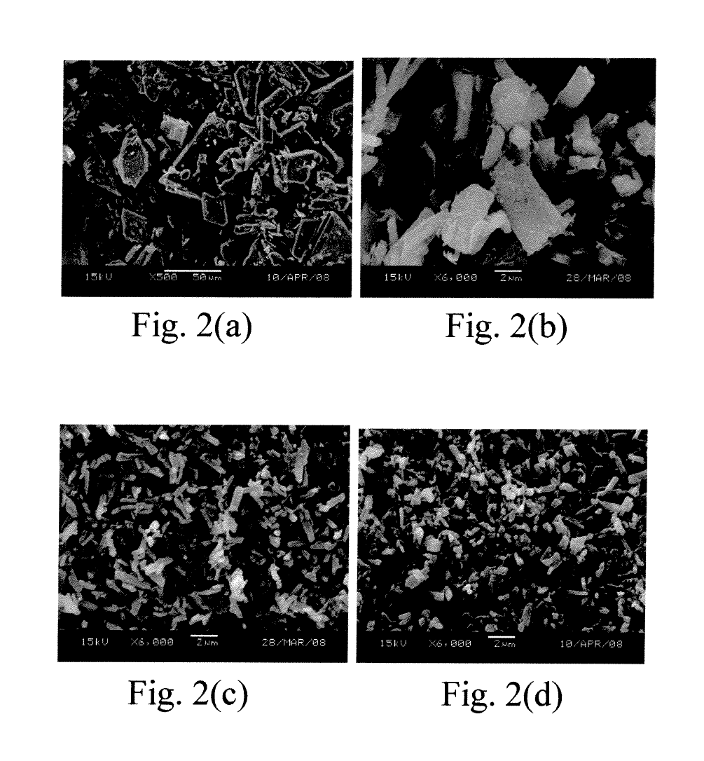

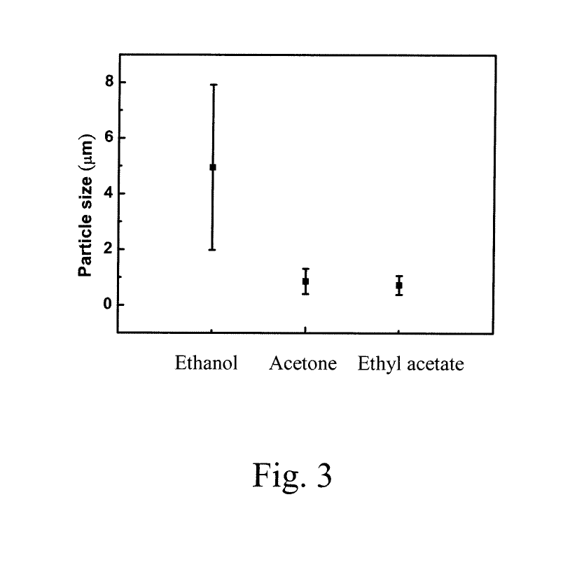

As shown in FIG. 2(a), the acetazolamide bulk drug has the crystal shape of the irregular lump shape. As shown in FIG. 2(b), the prepared acetazolamide microparticle of the embodiment 1 has an irregular crystal shape. As shown in FIGS. 2(c) and (d), the prepared acetazolamide microparticles of both the embodiments 2 and 3 have a rod-like crystal shape. FIG. 3 is a diagram showing the comparisons of the particle sizes and distributions between the embodiments 1 to 3. As shown in FIG. 3, when acetone and the ethyl acetate are used as the solvents, the micronization effect is better, wherein the ethyl acetate is the best solvent.

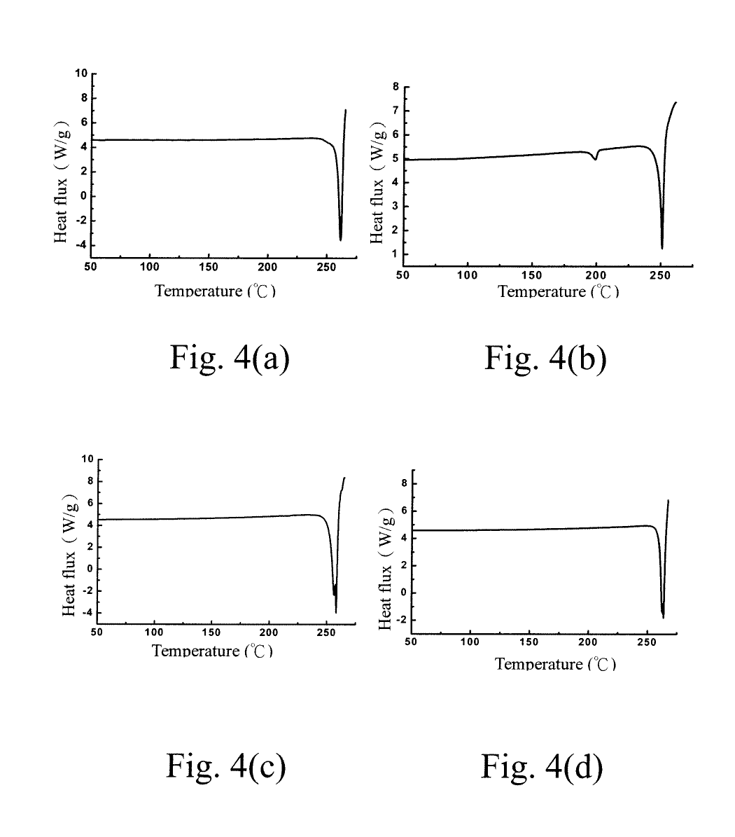

FIGS. 4(a)-(d) are diagrams showing the DSC analyses results of the acetazolamide bulk drug and the acetazolamide microparticles obtained from embodiments 1-3. As shown in FIG. 4(a), the acetazolamide bulk drug (crystal folin: Form II) has a melting point of 258-262° C. As shown in FIG. 4(b), the acetazolamide microparticle of the embodiment 1 has a melting point of 197-199° C. and a crystal form of Form I. However, with the increase of the temperature, the crystal form changes from Form I to Form II, and the melting point of the microparticles changes to 250-252° C. at the same time. As shown in FIGS. 4(c) and (d), embodiments 2 and 3 have a melting point of 256-259° C. and 262-264° C., respectively, and both have the crystal form of Form II. As to the crystal forms of the microparticles of the embodiments 1 to 3, the X-ray diffraction (XRD) patterns show the results the same as those indicated in FIGS. 4(a)-(d) (data not shown). Further, the FTIR patterns prove that no signal resulting from the solvent remaining on the microparticles is detected (data not shown).

### [Embodiments 4 to 9] Pressure and Temperature Effects

The operation parameters of the embodiments 4-9 are shown in Table 3.

TABLE 3

| | | solution | Conc. | | | mean | | |
|---|---|---|---|---|---|---|---|---|
| | | FR | (% of the | | | particle | | |
| embodiment | solvent | (ml/min) | sat. conc.) | T(° C.) | P(Pa) | size (μm) | SE(μm) | R(%) |
| 4 | ethyl | 1 | 30 | 35 | 100 | 0.73 | 0.34 | 60.81 |
| | acetate | | | | | | | |
| 5 | ethyl | 1 | 30 | 35 | 120 | 0.82 | 0.32 | 83.63 |
| | acetate | | | | | | | |
| 6 | ethyl | 1 | 30 | 35 | 140 | 1.04 | 0.49 | 84.66 |
| | acetate | | | | | | | |
| 7 | ethyl | 1 | 30 | 55 | 100 | 0.88 | 0.33 | 63.96 |
| | acetate | | | | | | | |
| 8 | ethyl | 1 | 30 | 55 | 120 | 0.90 | 0.37 | 59.37 |
| | acetate | | | | | | | |
| 9 | ethyl | 1 | 30 | 55 | 140 | 1.18 | 0.54 | 47.81 |
| | acetate | | | | | | | |

Abbreviations: FR, flow rate; Conc., concentration; Sat. conc., saturated concentration; T, temperature; P, pressure; SE, standard error; R, recovery rate.

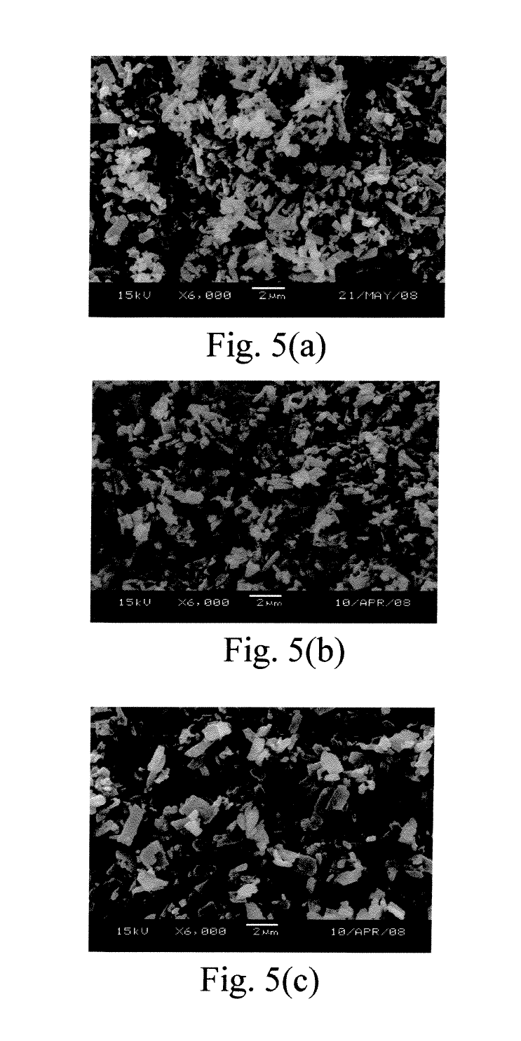

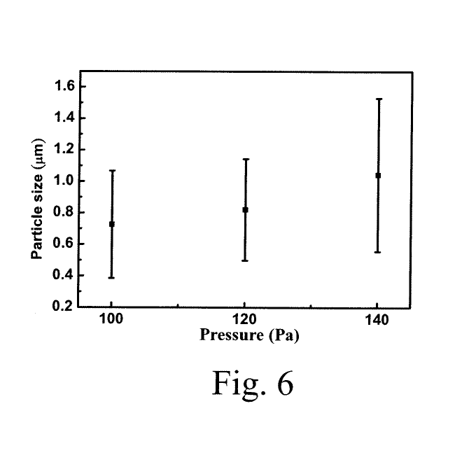

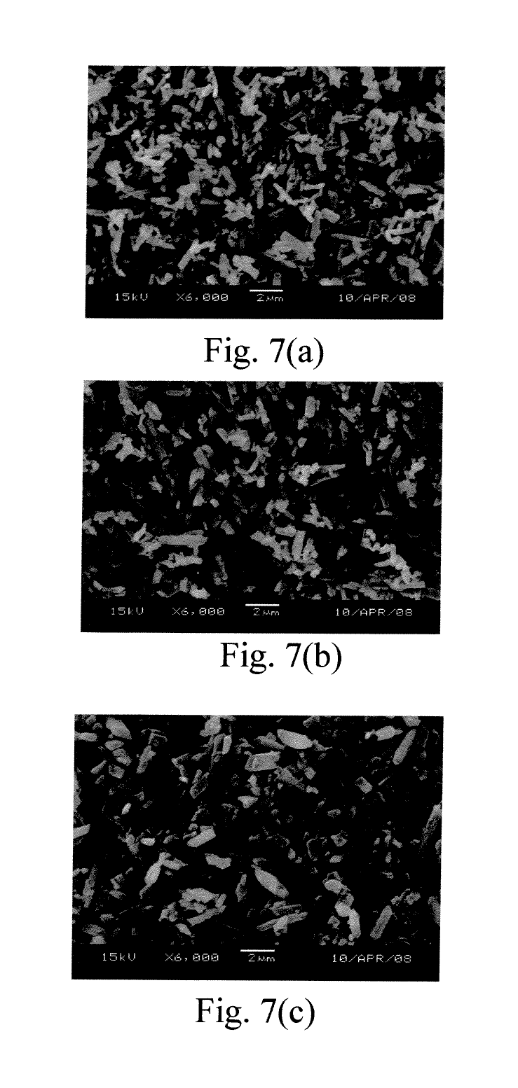

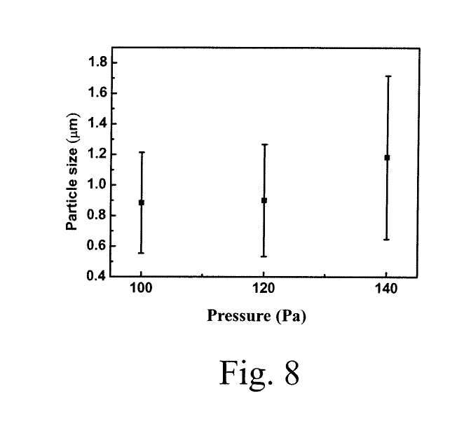

The pressure effect could be obtained by the comparison between the embodiments 4-6 at the fixed temperature 35° C. , and between the embodiments 7-9 at the fixed temperature 55° C. The embodiments 4-6 (as shown in FIGS. 5(a)-(c), respectively) and 7-9 (as shown in FIGS. 7(a)-(c), respectively) all have a rod-like crystal shape. FIGS. 6 and 8 are diagrams showing the comparisons of the particle sizes and distributions between the embodiments 4-6 and 7-9, respectively. As shown in FIGS. 6 and 8, either under the fixed temperature 35° C. or 55° C., the particle size increases with the increasing pressure.

As to the temperature effect, it could be known based on the comparisons between the embodiments 4 and 7, between the embodiments 5 and 8, and between the embodiments 6 and 9 that the particle sizes and distributions of the acetazolamide microparticles increase with the increasing temperature.

### [Embodiments 10 to 13] The Acetazolamide Solution Concentration and Flow Rate Effects

The operation parameters of the embodiments 10-13 are shown in Table 4.

TABLE 4

| | | solution | Conc. | | | mean | | |
|---|---|---|---|---|---|---|---|---|
| | | FR | (% of the | | | particle | | |
| embodiment | solvent | (ml/min) | sat. conc.) | T(° C.) | P(Pa) | size (μm) | SE(μm) | R(%) |
| 10 | ethyl | 1 | 30 | 35 | 100 | 0.73 | 0.34 | 60.81 |
| | acetate | | | | | | | |
| 11 | ethyl | 1 | 90 | 35 | 100 | 0.36 | 0.12 | 36.17 |
| | acetate | | | | | | | |
| 12 | ethyl | 2 | 30 | 35 | 100 | 2.96 | 1.90 | 28.84 |
| | acetate | | | | | | | |
| 13 | ethyl | 2 | 90 | 35 | 100 | 2.83 | 2.07 | 70.74 |
| | acetate | | | | | | | |

Abbreviations: FR, flow rate; Conc., concentration; Sat. conc., saturated concentration; T, temperature; P, pressure; SE, standard error; R, recovery rate.

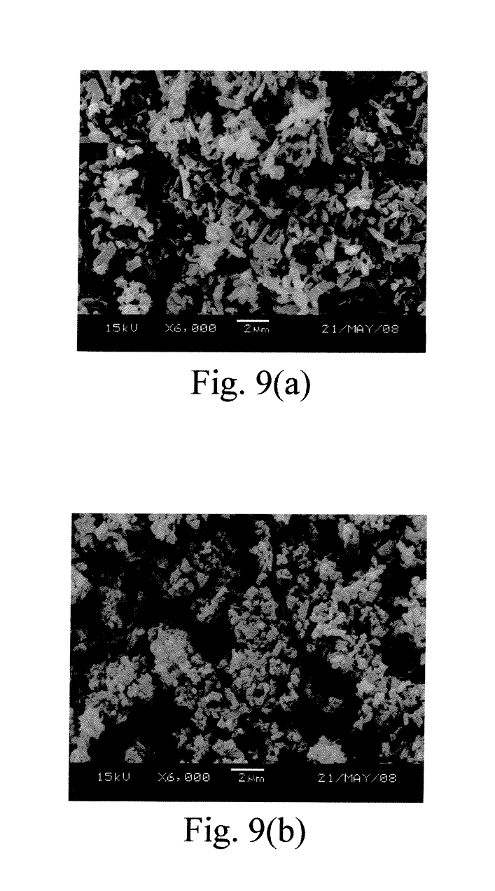

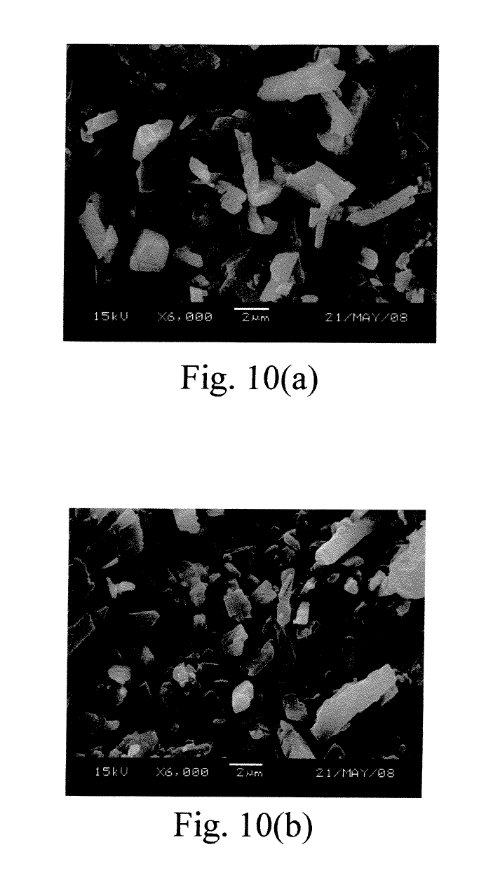

The solution concentration effect could be obtained by the comparisons between the embodiments 10 and 11 at the fixed flow rate 1 ml/min, and between the embodiments 12 and 13 at the fixed flow rate 2 ml/min. The embodiments 10, 11 and 13 (as shown in FIGS. 9(a)-(b) and 10 (b), respectively) all have a rod-like crystal shape, and the embodiment 12 (as shown in FIG. 10(a)) has an irregular crystal shape. Based on the above, it could be known that the particle size and distribution decreases with the increasing solution concentration.

As to the effect of the flow rate of the acetazolamide solution, based on the comparisons between the embodiments 10 and 12 and between the embodiments 11 and 13, it could be known that the particle sizes and distributions of the acetazolamide microparticles increase with the increasing solution flow rate.

Based on the embodiments 1-13, the optimal condition for preparing the acetazolamide microparticles of the present invention by the continuous SAS method is as follows: solvent: ethyl acetate, pressure: 100 Pa, temperature: 35° C., acetazolamide solution concentration: 90% of the saturated concentration and the acetazolamide solution flow rate: 1 ml/min. Via the optimal condition, the acetazolamide microparticle with a mean particle size of 0.36±0.12 μm could be obtained.

[Dissolution Rate Analysis]

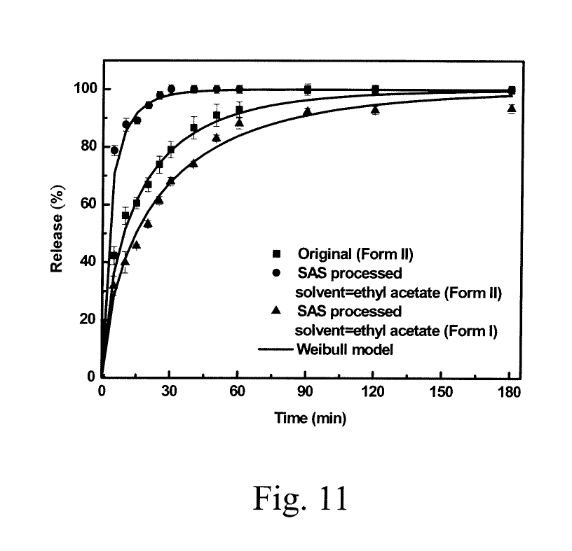

FIG. 11 is a curve diagram showing the dissolution rate of the acetazolamide bulk drug, embodiments 11 and 1, wherein both the acetazolamide bulk drug (shown as the “original (Form II)”) and the embodiment 11 have the crystal form of Form II and the embodiment 1 has the crystal form of Form I. As shown, the acetazolamide microparticle prepared under the optimal condition (embodiment 11) has an apparently increased dissolution rate, and that prepared by the ethanol solvent (embodiment 1) has a slower dissolution rate. When the Weibull model is adopted to describe the dissolution rate of the acetazolamide, the dissolution rate coefficients of the acetazolamide bulk drug, embodiment 1 and embodiment 11 are 0.0626 min⁻¹, 0.2745 min⁻¹ and 0.0399 min⁻¹, respectively. Therefore, compared with the acetazolamide bulk drug, the acetazolamide microparticle of the embodiment 11 has a about 4.4-fold increased dissolution rate, but that of the embodiment 1 has a about 0.64-fold decreased dissolution rate.

While the invention has been described in terms of what is presently considered to be the most practical and preferred embodiments, it is to be understood that the invention needs not be limited to the disclose embodiments. Therefore, it is intended to cover various modifications and similar arrangements included within the spirit and scope of the appended claims, which are to be accorded with the broadest interpretation so as to encompass all such modifications and similar structures.

What is claimed is:

1. An acetazolamide microparticle having a mean particle size ranged between 0.36 μm and 18 μm.

2. An acetazolamide microparticle as claimed in claim 1, wherein the mean particle size is smaller than 10 μm.

3. An acetazolamide microparticle as claimed in claim 1, wherein the mean particle size is smaller than 5 μm.

4. An acetazolamide microparticle as claimed in claim 1, wherein the mean particle size is smaller than 1 μm.

5. An acetazolamide microparticle as claimed in claim 1, having a crystal form being one of Form I and Form II.

6. An acetazolamide microparticle as claimed in claim 1, having a rod-like crystal shape.

7. A method for preparing an acetazolamide microparticle having a mean particle size ranged between 0.36 μm and 18 μm, comprising steps of:
dissolving an acetazolamide in a solvent to form an acetazolamide solution; and
mixing the acetazolamide solution with a supercritical fluid at a temperature and a pressure above a critical point of the supercritical fluid for foaming the acetazolamide microparticle, wherein the solvent is miscible with the supercritical fluid.

8. A method as claimed in claim 7, wherein the solvent is selected from a group consisting of a methanol, an ethanol, a methylene chloride, an N-methyl-pyrrolidone (NMP), an ethyl acetate, an acetone and a combination thereof.

9. A method as claimed in claim 8, wherein the solvent is the ethyl acetate.

10. A method as claimed in claim 7, wherein the acetazolamide solution has one of concentrations equal to and higher than 25% of a saturated concentration.

11. A method as claimed in claim 10, wherein the concentration of the acetazolamide solution is one of concentrations equal to and higher than 50% of the saturated concentration.

12. A method as claimed in claim 10, wherein the concentration of the acetazolamide solution is one of concentrations equal to and higher than 75% of the saturated concentration.

13. A method as claimed in claim 7, wherein the supercritical fluid is a supercritical carbon dioxide serving as a supercritical anti-solvent (SAS).

14. A method as claimed in claim 7, wherein the mixing step comprises a step of delivering the acetazolamide solution into a container containing the supercritical fluid at a flow rate of 0.1 to 5 ml/min.

15. A method as claimed in claim 14, wherein the flow rate of the acetazolamide solution is ranged from 0.8 to 1.5 ml/min.

16. A method as claimed in claim 7, wherein the pressure of the supercritical fluid is in a range between 80 Pa and 160 Pa and the temperature is in a range between 20° C. and 70° C.

17. A method as claimed in claim 16, wherein the pressure is in a range between 90 Pa and 110 Pa and the temperature is in a range between 30° C. and 45° C.

18. A method as claimed in claim 7, further comprising a purification step for removing the solvent remaining on the acetazolamide microparticle.

19. A method as claimed in claim 18, wherein the purification step comprises a step of delivering the supercritical fluid onto the acetazolamide microparticle.

20. A method for treating a disease being one selected from a group consisting of a diuresis, a high ocular pressure, a glaucoma, a high altitude disease, an epilepsy and an edema, comprising a step of administering to a subject in need thereof a pharmaceutical composition comprising an acetazolamide microparticle having a mean particle size ranged between 0.36 μm and 18 μm.
